(19) United States
(12) Patent Application Publication
(10) Pub. No.: US 2026/0335578 A1
(43) Pub. Date: Oct. 29, 2026

(54) METHOD AND SYSTEM FOR EXPIRATORY HIGH FREQUENCY PERCUSSIVE VENTILATION

(71) Applicants: POLITECNICO DI MILANO, Milano (IT); UNIVERSITY OF SZEGED, Szeged (HU)

(72) Inventors: Raffaele Dellaca, Milano (IT); Ferenc Petak, Szeged (HU); Walid Habre, Geneva (CH)

(21) Appl. No.: 19/107,807

(22) PCT Filed: Sep. 1, 2023
(86) PCT No.: PCT/EP2023/074077

§ 371 Date: Feb. 28, 2025

(30) Foreign Application Priority Data
Sep. 2, 2022 (IT) 102022000018081

(51) Int. Cl. A61M 16/00; A61M 16/08; A61M 16/20

(52) U.S. Cl. CPC A61M 16/024; A61M 16/0003; A61M 16/0063; A61M 16/0883; A61M 16/203; A61M 2016/0018; A61M 2016/0039; A61M 2016/0042; A61M 2202/0007; A61M 2202/02; A61M 2205/3303; A61M 2205/3327; A61M 2205/3334; A61M 2205/3344; A61M 2205/50; A61M 2210/1025; A61M 2230/005; A61M 2230/08; A61M 2230/40; A61M 2230/63

(57) ABSTRACT

The method and system according to preferred embodiments of the present invention provides improved method and systems for mechanical ventilation. A system and method are provided for delivering expiratory percussive ventilation/high frequency expiratory percussive ventilation for patients with healthy lungs, with respiratory disorders, such as adverse hypoxia/hypercapnia encountered in obesity, bariatric surgery, or surgeries requiring capnoperitoneum. The method and system according to embodiments of the present invention are also useful to assist with lung diseases frequently encountered in mechanically ventilated patients, such as acute respiratory distress syndrome (ARDS) or ventilator-induced lung injury (VILI).

### FIELD OF TECHNOLOGY

The present invention relates to the field of devices and systems for mechanical ventilation, more particularly for delivering lung protective ventilation for patients needing respiratory support.

### BACKGROUND

Mechanical ventilation is normally needed when patients cannot, for several reasons, breath autonomously. In particular, for patients requiring critical care to facilitate appropriate oxygen supply and carbon dioxide removal, e.g. those under general anaesthesia or intensive care, Intermittent Positive Pressure Ventilaton (IPPV) technique is regularly applied. This technique conventionally consists in the application of two different level of pressure, a higher pressure called Peak Inspiratory Pressure (PIP) and a lower one called Positive End Expiratory Pressure (PEEP), for producing an artificial breath. The increased pressure produces an inspiration, i.e. the breathing gas is moved from outside into the lung, while the lower pressure allows the lung to exhale thanks to the pressure due to the elastic recoil of the respiratory system that produces the expiration. Although maintaining physiologic lung ventilation is feasible in patients with healthy lungs, different pulmonary disorders pose challenges to health care providers when ensuring adequate gas exchange during ventilatory support. Particularly, diseases with decreased functional residual capacity and/or decreased lung compliance, as observed e.g. in patients with obesity or in those with acute respiratory distress syndrome (ARDS), require continuous adjustment of the ventilation strategy to maintain appropriate gas exchange. In addition, gas exchange abnormalities during mechanical ventilation are observed in clinical conditions associated with significant lung restriction and compromised gas exchange, which include laparoscopic surgery in patients with capnoperitoneum and bariatric surgery.

All conditions associated to lung volume de-recruitments, decreased lung tissues of chest wall compliance, or increased peripheral airway resistance (including those listed above) the provision of an adequate gas exchange may lead to excessive regional lung tissue stress, as the tidal volume required for ventilation inflates only a fraction of the lung. This excessive stress may damage the lung and promote inflammatory response leading to the so-called Ventilator-Induced Lung Injury (VILI) which, despite considerable improvements in respiratory management, continue to pose a burden on health care. Thus, ventilation support modalities must be improved with the aim of ensuring adequate lung ventilation without causing excessive stress and strain on lung tissues. A possible solution for improving gas exchange is represented by High-frequency percussive ventilation (HFPV) based on the combination of conventional and high-frequency oscillatory ventilation. HFPV applies a pressure waveform by over-imposing to a conventional (IPPV) mechanical ventilation pressure waveform a high-amplitude and high frequency pressure oscillation pattern. See for example:

Allan P F, Osborn E C, Chung K K, and Wanek S M. High-frequency percussive ventilation revisited. J Burn Care Res 31:510-520, 2010; Butler A D, Dominick C L, and Yehya N. High frequency percussive ventilation in pediatric acute respiratory failure. Pediatr Pulmonol 56:502-508, 2021; Chung K K, Wolf S E, Renz E M, Allan P F, Aden J K, Merrill G A, Shelhamer M C, King B T, White C E, Bell D G, Schwacha M G, Wanek S M, Wade C E, Holcomb J B, Blackbourne L H, and Cancio L C. High-frequency percussive ventilation and low tidal volume ventilation in burns: a randomized controlled trial. Crit Care Med 38:1970-1977, 2010; Derdak S, Mehta S, Stewart T E, Smith T, Rogers M, Buchman T G, Carlin B, Lowson S, Granton J, and Multicenter Oscillatory Ventilation For Acute Respiratory Distress Syndrome Trial Study I. High-frequency oscillatory ventilation for acute respiratory distress syndrome in adults: a randomized, controlled trial. Am J Respir Crit Care Med 166:801-808, 2002; Godet T, Jabaudon M, Blondonnet R, Tremblay A, Audard J, Rieu B, Pereira B, Garcier J M, Futier E, and Constantin J M. High frequency percussive ventilation increases alveolar recruitment in early acute respiratory distress syndrome: an experimental, physiological and CT scan study. Crit Care 22:3, 2018; Korzhuk A, Afzal A, Wong I, Khusid F, Worku B, and Gulkarov I. High-Frequency Percussive Ventilation Rescue Therapy in Morbidly Obese Patients Failing Conventional Mechanical Ventilation. J Intensive Care Med 35:583-587, 2020. This modality (HFPV) has the combined benefits of conventional tidal expansions that maintain the lung open and produces tidal volumes for producing alveolar ventilation by convective gas transport and high-frequency fluctuations that facilitate axial gas mixing by enhancing gas diffusion mechanisms for facilitating gas transport. However, despite improvement in gas exchange, this technique presents the drawback that superposition of the high frequency component to the conventional ventilation during inspiration leads to higher peak pressures that may lead tissue overdistension at the level of the airways and alveoli, especially in very low-compliance patients. Furthermore, increased peak pressure during HFPV, adversely affects hemodynamics via compromising venous return. All these drawbacks of HFPV require to keep lower tidal volumes for the conventional ventilation waveform or lower peak-to-peak high frequency components to avoid Ventilation Induced Lung Injury (VILI). These drawbacks generally limit the net benefit of this ventilation mode and can be a serious problem in case of severe ARDS patients.

An improved method and system for mechanical ventilation which is capable of exploiting the advantages of HFPV without the drawbacks mentioned above would have a great value in improving patient care under general anaesthesia and intensive care or, in general, for patients requiring respiratory support.

### Objects of the Invention

It is an object of the present invention to provide a system and method which alleviates the drawbacks associated with the prior art.

### SUMMARY OF THE INVENTION

The present invention provides a method and system as set out in the accompanying claims.

According to one aspect of the present invention, we provide a system for mechanical ventilation of a human patient, including: pressure generator means for generating a pressure or flow of breathing gasses to produce ventilation waveforms; interface means for delivering the ventilation to a patient, including a patient interface and a breathing circuit, the breathing circuit connecting the pressure generator means to the patient interface; detecting means adapted to detect the inspiration/expiration phases of the patient's breathing cycle and to determine a first value indicative of the airflow entering the breathing circuit and a second value indicative of the airflow at the patient's airway opening; a microprocessor adapted to receive information from the detecting means and to control the generator means; characterized in that the microprocessor is configured to control the pressure generator means to provide a high frequency ventilation to the patient only during the expiration phase and to maintain, during expiration phase, the rate of flow entering the breathing circuit equal or greater than the peak flow exiting the patient's airway opening for allowing purging of exhaled breath at the patient interface.

In a possible embodiment, the microprocessor is further configured to control the pressure generator means to produce a conventional ventilation pattern to ventilate the patient during the inspiratory phase and to superimpose a high frequency ventilation patter on the conventional ventilation pattern to ventilate the patient during the expiratory phase, together with the purging flow. In a preferred embodiment the pressure generator means include: a flow generator configured to produce a ventilation waveform; and a high frequency oscillatory pressure generator configured to generate high frequency oscillatory pressure; the microprocessor being configured to control the flow generator and a high frequency oscillatory pressure generator to provide a combined ventilation pattern where a high frequency ventilation pattern is superimposed on the conventional ventilation pattern to ventilate the patient during the expiratory phase.

The generation of high frequency oscillatory pressure can be done with one or more of the following technique: piston/voice coil actuator system, high speed proportional gas valves, high dynamic radial compressor, pneumatic or jet systems.

The detecting means can include one or more of the following: airway opening of patient's circuit pressure sensor, patient's flow sensor, Graseby capsule to detect movements of the patient's thoraco-abdominal wall, sensor to detect electrical signals of patient's diaphragmatic activity, breathing circuit input flow sensors, breathing circuit outlet flow sensors, devices for measuring the pressure generator operating conditions that allows determining the gas flow entering the breathing circuit (such as differential pressure and rotational speed for blowers, shaft position and differential pressure for proportional valves).

In a preferred embodiment, the conventional ventilation includes one or more of the following technique: controlled mechanical ventilation; assisted mechanical ventilation; assisted mechanical ventilation with trigger systems for detecting the patient spontaneous activity and synchronize the delivery of the inspiratory or expiratory pressure to the patient's spontaneous breaths.

The breathing circuit can optionally include an inspiratory line and a separated exhalation line. According to an alternative embodiment, the breathing circuit can include a single tube and an intentional leak allowing gas purging at the patient's airway opening during expiratory phase.

In a second aspect of the invention we provide a method for operating the system of any preceding claim, comprising the step of: detecting by means of the at least one sensor the alternation of inspiration and expiration phases in a patient; controlling the high frequency oscillatory pressure generator to provide a High Frequency Ventilation overimposed to the conventional ventilation pattern to the patient only during the expiration phase.

A still further aspect of the present invention provides a computer program for controlling the method described above.

Also a therapeutic method is provided for treating patients suffering respiratory failures with the system described above.

The method and system according to preferred embodiments of the present invention applies high-frequency component during the expiration phase only, thus maintaining the benefit of increased diffusion gas exchange as for percussive ventilation without increasing the peak inspiratory pressure applied to the patients, thereby providing protection against excessive stress and strain to lung and airway tissue. Producing a ventilation mode according to the present invention, combines the advantages of conventional mechanical ventilation and high-frequency mechanical ventilation without the drawbacks of percussive ventilation. The system produces a conventional ventilation pattern during the inspiratory phase delivering a PIP to the patients, e.g. with the modes currently used in mechanical ventilators (fast pressure rise, ramp pressure increase, etc) for the duration of the inspiration. During the expiratory phase the system superimposes a high-frequency oscillatory ventilation pattern.

The system and method (including the therapeutic method) of the present invention can be very useful for several kind of patients, including patients with healthy lungs, with respiratory disorders, such as adverse hypoxia/hypercapnia encountered in chronic respiratory conditions (COPD, chest wall or lung tissue restrictive disorders, neuromuscular diseases), obesity, bariatric surgery, or surgeries requiring capnoperitoneum. The method and system of the present invention is also useful for lung diseases frequently encountered in mechanically ventilated patients who suffer respiratory failures, such as Acute Respiratory Failure (ARF), Acute Lung Injury (ALI), Acute Respiratory Distress Syndrome (ARDS) or Ventilator-Induced Lung Injury (VILI).

### BRIEF DESCRIPTION OF THE DRAWINGS

Reference will now be made, by way of example, to the accompanying drawings, in which:

FIG. 1 shows a comparison between traditional mechanical ventilation and the expiration Percussive High Frequency Percussive Ventilation (eHFPV) according to a preferred embodiment of the present invention: the graph shows tracheal pressure signals during application of conventional pressure-controlled ventilation (PCV) and expiratory High Frequency Percussive Ventilation (e-HFPV) in a rabbit by adding 5 Hz pressure fluctuations with 2 cmH₂ O peak-to-peak pressure. ;

FIG. 2 is a schematic diagram of the system implementing a preferred embodiment of the present invention

FIGS. 3a and 3b show possible embodiment of the breathing circuit according to the present invention;

FIG. 4 schematically shows an alternative embodiment, using a chest cuirass for delivering the pressure oscillations to the patient.

### DETAILED DESCRIPTION OF PREFERRED EMBODIMENTS

Traditional High-frequency Percussive Ventilation (hfpv) Is Based on the superposition of a high-frequency oscillatory pressure signal (usually from 5 to 15 Hz) over the pressure regimen during conventional mechanical ventilation (CMV), with the oscillatory component, i.e. the High Frequency Ventilation (HFV), continuously applied during the breathing cycle. While this approach improves tissue oxygenation and CO₂ clearance, it implies the application of higher inspiratory pressures, thereby increasing the risk of excessive stress and strain to the lung parenchyma and to the bronchial wall.

According to our invention, the high-frequency oscillatory component is applied only during the expiratory phase of the conventional waveform. In this so-called Expiratory High Frequency Percussive Ventilation (e-HFPV) modality, the synchronized high-frequency oscillation is expected to have similar benefits to the HFPV in terms of gas exchange with more protective against ventilation-induced lung injury (VILI).

According to a preferred embodiment, to implement this approach (i.e. a high frequency pressure fluctuations during expiration only), the pressure and/or flow will be measured at the airway opening of the patient, and data processing algorithm will detect the beginning and the end of the expiratory phase, whenever determined by the patient (assisted ventilation modes) of by the ventilator (controlled ventilation modes). This signal will be used to enable the high-frequency pressure generator that will be activated only during expiration. In a preferred embodiment, the resulting ventilation waveform is the combination of a conventional ventilation and the superimposed high-frequency component during expiration. The high-frequency component will be preferable delivered between 5 and 15 Hz with peak-to-peak pressure greater than 2 cmH₂ O, with maximum airway opening pressure during expiration preferably not exceeding patient's specific PIP. The high frequency oscillatory pressure and the conventional ventilation pressure may be generated by proper controlling a single high speed actuator or, alternatively, by two different devices, controlled by a microprocessor, each one generating a different kind of ventilation. In any case, during inspiratory phase, only the conventional ventilation is delivered to the patient, while, during expiratory phase a combination of conventional and high pressure ventilation is provided with the high frequency wave form being superimposed on the conventional wave form.

In order to promote gas exchange, it is important that the inlet of the patient respiratory system is always exposed to fresh respiratory gases, in order to maximise the concentration gradients of oxygen and carbon dioxide between patients' alveoli and airway opening. As the high frequency oscillations are boosting gas diffusion but also producing higher gas flows in and out of the patient's respiratory system, it is necessary to wash out from the breathing circuit not only the tidal expiratory flow but also that generated by the high frequency oscillations. For this reason, a flow of fresh respiratory gas equal or larger than the sum of the peak oscillatory flow and the peak tidal expiratory flow must be over-imposed to the ventilation flow as close as possible to the airway opening. This flow allows purging the exhaled carbon dioxide and restoring the proper inspiratory fraction of oxygen, improving effectiveness of the respiratory support.

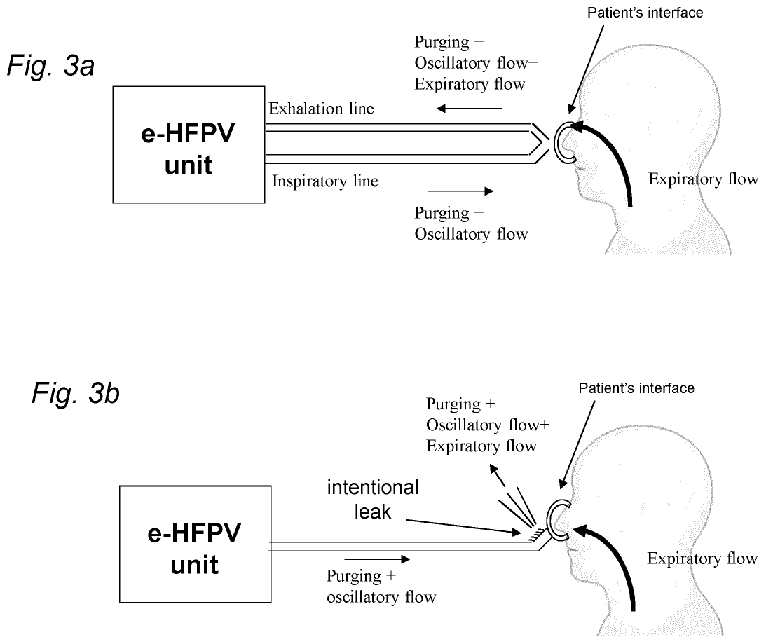

This can be achieved with different approaches, depending of the type of the breathing circuit in use. For double-limb ventilation circuits, the ventilator needs to measure or estimate the flow entering the circuit and the maximum expiratory flow resulting by the combination of patient's tidal breathing and that resulting from the application of the high frequency pressure oscillations (maximum overall exhaled flow, MOEF) and set a continuous purging flow entering the inspiratory line larger than this value (FIG. 3a). When a single limb ventilation circuit is used, the purge flow is obtained by placing an intentional leak nearby the patient interface (i.e. nasal or full-face mask, nasal prongs, tracheal tubes, tracheostomy tubes, etc) (FIG. 3b). In this case the purged flow can be estimated from the pressure of the gas in the breathing circuit after a proper characterisation of the pressure-flow relationship of the intentional leak. The design of the intentional leak should be done considering the expected range of MOEF and the minimum circuit pressure during expiration, known as Positive End Expiratory Pressure (PEEP). During ventilator operations, PEEP should be limited to values equal or larger than those providing a leaked flow equal to the MOEF.

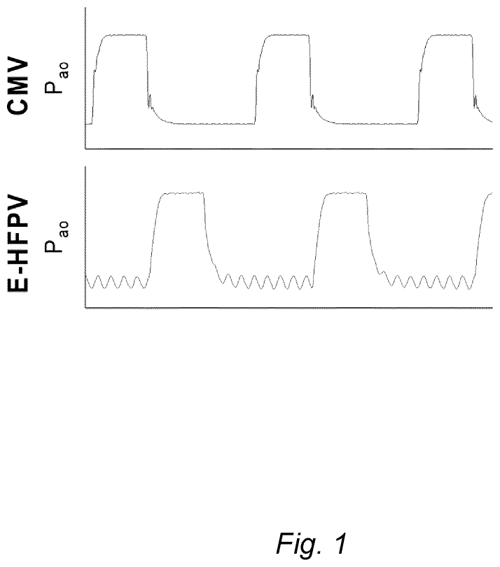

Represented in FIG. 1 are two graphs showing the comparison of e-HFPV method according to a preferred embodiment of the present invention with a whole cycle HFPV and conventional mechanical ventilation (CMV). In the example shown in FIGS. 1, 5 Hz fluctuations with 2 cmH2O peak-to-peak pressure was superimposed during mechanical ventilation in an anesthetized animal model.

One of the advantages of implementing eHFPV is to enhance CO₂ clearance and to increase oxygenation by facilitating dynamic diffusion without overdistending the lungs. Indeed, our results confirmed the improvements in gas exchange when a high-frequency signal with an amplitude of 2 cmH₂O and frequency of 5 Hz was applied in the expiratory phase only. The improved CO₂ clearance was also confirmed by the capnography results demonstrating enhancements in V_{CO2} at all amplitudes and frequencies during eHFPV (p<0.001), and improvements in ventilation dead space (VDF/VT) and phase 2 slope (S2V) at higher oscillatory amplitudes.

Compared to the already available HFPV, application of a method according to the present invention avoids the application of high-frequency oscillation in the inspiratory phase, limiting the maximum pressure applied to the lungs. This is confirmed by the lack of an increase in peak airway pressure during e-HFPV while delivering the same tidal volume VT as the HFPV or the conventional CMV. Due to this benefit of e-HFPV, this modality is expected to protect lung tissue injury since the lung compliance is minimal at end-inspiration, causing the lung tissues to be more prone to injure in this phase of the breathing cycle.

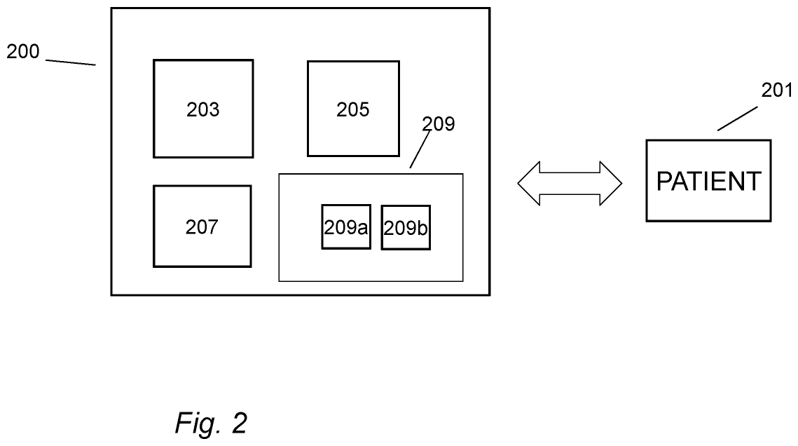

FIG. 2 represents a block diagram of a device to implement the e-HFPV ventilation according to a preferred embodiment of the present invention. The system (device) 200 for mechanical ventilation of a human patient 201, includes a flow or pressure generator 203, configured to produce a ventilation pattern and a high frequency oscillatory pressure generator 205 configured to generate a High Frequency oscillatory pressure signal to improve gas diffusion processes within the patients' lung. Also a controller (e.g. a microprocessor) 207 is provided which controls the operation of the flow or pressure generator 203 and the high frequency oscillatory pressure generator 205. The controller 207 receives input from detecting means 209 (e.g. including a pressure sensor 209a) which allows the controller to determine when the patient is in an inspiration phase and when the patient is in an expiration phase; also the detecting means are adapted to determine a first value indicative of the airflow entering the breathing circuit and a second value indicative of the airflow at the airway opening to the patient. According to such information, the microprocessor controls the high frequency oscillatory pressure generator to provide a High Frequency Percussive Ventilation to the patient only during the expiration phase. During the inspiration phase the system according a preferred embodiment of the present invention provides the patient with a conventional ventilation delivering a PIP to the patients with the modes currently used in mechanical ventilators (fast pressure rise, ramp pressure increase, etc) for the duration of the inspiration. As mentioned above, in some embodiments the HFO generator may be obtained properly controlling the inspiratory and PEEP valves of the ventilator or, in case of turbine-based ventilators, by controlling the blower, if such components are designed to be fast enough to deliver high frequency oscillations. Therefore it is possible that a single device for generating a ventilation pattern replaces the two separate devices 203 and 205. In this case it is the microprocessor 207 which is configured to single pressure and flow generator to provide a combined ventilation pattern where a high frequency ventilation pattern is superimposed on the conventional ventilation pattern to ventilate the patient during the expiratory phase. The same processor is also using measures or estimations of the patient's flow and the breathing circuit input flow for assuring the presence of the purge gas flow described above.

These measures/estimations can be obtained by one or more of the following systems (only two systems, 209a and 209b, are represented in FIG. 2, but they could be more than two systems or devices (209c, 209d . . . )): airflow sensor at the outlet of the pressure/flow generation means; sensors providing the status of the pressure/airflow generation means that can be used for estimating the airflow, such as differential pressure and rotational speed when radial compressors are used or valve shaft position sensors and differential pressure when proportional valves are used; airflow sensors at the exhalation port of the ventilation system that, combined with patient airway opening flow, can provide the estimation of the gas flow entering the breathing circuit (gas flow entering the breathing circuit+patient airway opening flow=airflow sensors at the exhalation port of the ventilation system); pressure sensor and the knowledge of the pressure/flow relationship for estimating the exhalation port flow of an intentional leak component when single-limb ventilation circuits are used and using this information as described in the previous approach. The at least one mean for measuring the airway opening flow of the patient can include one or more of the following: airflow sensor at the patient's interface (eventually combined with leaks estimation algorithms, especially during non-invasive ventilation); gas flow sensor at the exhalation port of the ventilation system that, combined with gas flow sensors at the inlet of the breathing circuit, can provide the estimation of the gas flow at the airway opening of the patient (gas flow entering the breathing circuit+patient airway opening flow=airflow sensors at the exhalation port of the ventilation system); pressure sensor and the knowledge of the pressure/flow relationship for estimating the exhalation port flow of an intentional leak component when single-limb ventilation circuits are used and using this information as described in the previous approach.

FIG. 3a shows the breathing circuit and the different gas flows determining the purge flow necessary for assuring the maximal carbon dioxide and oxygen concentration gradients during expiration in e-HFPV ventilation systems. The pressure/flow generator feeds fresh respiratory gasses into the inspiratory line even during the expiratory phase, assuring that this flow is larger than the MOEF. In this conditions, the oscillatory exhaled flow is immediately removed at the inlet of the patient's interface, maximising the concentration gradient between the alveoli and the airway opening of the patient. If this condition is fulfilled, there will always be flow exiting the system by the expiratory line and exhalation port of the ventilator.

FIG. 3b shows an alternative breathing circuit which uses only one tube connecting the patient to the e-HFPV ventilation unit. Also in this figure, the definition of the purge flow necessary for assuring the maximal carbon dioxide and oxygen concentration gradients during expiration in e-HFPV ventilation systems is shown. The pressure/flow generator feeds fresh respiratory gasses into the breathing line and the presence of a mechanical intentional leak at the inlet of the patient interface of within the patient interface itself produces a continuous exhaled flow throughout each full breathing cycle, therefore even during the expiratory phase, assuring gas purging at the patient's airway opening. The amount of purging flow can be estimated from the measurement of the pressure of the gas in the breathing circuit, after the characterisation of the pressure-flow relationship of the intentional leak.

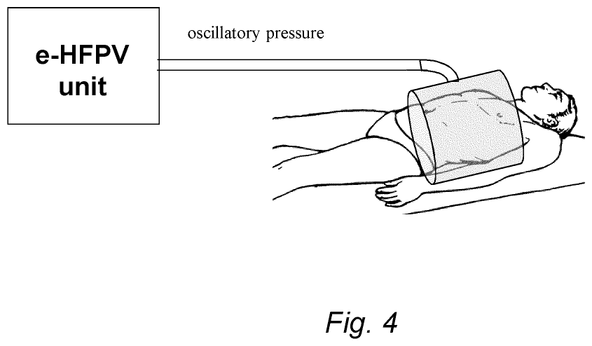

According to a possible alternative, represented in FIG. 4, the pressure oscillations are applied at the patient's chest wall by using a chest cuirass instead of applying them at the airway opening, eventually in combination with a standard mechanical ventilator providing either continuous positive airway pressure or whichever intermittent positive pressure ventilation mode at the airway opening of the patient.

As represented in FIG. 4, the ventilation support system may be applied, alternatively, also to the second port of the respiratory system, i.e. the chest wall surface. In this case, the patient's interface is made by a chest cuirass or an iron lung, connected to the e-HFPV device which applies the oscillatory pressure to the chest wall surface and, consequently, to the alveoli, producing an oscillatory flow at the airway opening (FIG. 4). In this case, the use of inverted direction of the pressure (negative swings instead of positive) and/or the over-imposition of a continuous negative expiratory pressure (CNEP) to the oscillatory pressures promotes and maintains lung volume recruitment. This system can be applied either by itself or in combination to positive pressure ventilators connected to the airway opening. In this last configuration, the ventilator connected to the airway opening must assure the presence of the purge flow as described above.

A test has been performed to validate the above described hypothesis, we compared the novel mechanical ventilation pattern expiratory high-frequency percussive ventilation (eHFPV) to a standard whole-cycle HFPV and conventional mechanical ventilation. Moreover, whether e-HFPV improves oxygen delivery and carbon dioxide (CO₂) clearance in an experimental model of induced hypoxia and hypercapnia was evaluated. To assess the extent of generalizing our findings to other species, a simulation study was performed to investigate the effects of lung size on oscillatory pressure transmission from the airway opening to the alveolar compartment.

1. A system for mechanical ventilation of a human patient, including;
pressure generator means for generating a pressure or flow of breathing gasses to produce ventilation waveforms;
interface means for delivering the ventilation to a patient, including a patient interface and a breathing circuit, the breathing circuit connecting the pressure generator means to the patient interface;
detecting means adapted to detect inspiration/expiration phases of the patient's breathing cycle and to determine a first value indicative of the airflow entering the breathing circuit and a second value indicative of the airflow at the patient's airway opening; and
a microprocessor adapted to receive information from the detecting means and to control the pressure generator means;
characterized in that the microprocessor is configured to control the pressure generator means to provide a high frequency ventilation to the patient only during the expiration phase and to maintain, during expiration phase, the rate of flow entering the breathing circuit equal or greater than the peak flow exiting the patient's airway opening for allowing purging of exhaled breath at the patient interface.

2. The system of claim 1, wherein the microprocessor is configured to control the pressure generator means to produce a conventional ventilation pattern to ventilate the patient during the inspiratory phase and to superimpose a high frequency ventilation pattern on the conventional ventilation pattern to ventilate the patient during the expiratory phase.

3. The system of claim 2, wherein the pressure generator means include:
a flow or pressure generator configured to produce a ventilation waveform; and
a high frequency oscillatory flow or pressure generator configured to generate high frequency oscillatory pressure;
the microprocessor being configured to control the flow or pressure generator and the high frequency oscillatory flow or pressure generator to provide a combined ventilation pattern where a high frequency ventilation pattern is superimposed on the conventional ventilation pattern to ventilate the patient during the expiratory phase.

4. The system of claim 3 wherein the high frequency oscillatory flow or pressure generator includes one or more of the following: a piston/voice coil actuator system, high speed proportional gas valves, a high dynamic radial compressor, pneumatic or jet systems.

5. The system of claim 1 wherein the detecting means includes one or more of the following: airway opening of patient's circuit pressure sensor, patient's flow sensor, Graseby capsule to detect movements of the patient's thoraco-abdominal wall, sensor to detect electrical signals of patient's diaphragmatic activity, breathing circuit input flow sensors, breathing circuit outlet flow sensors, devices for measuring the pressure generator means operating conditions that allows determining the gas flow entering the breathing circuit.

6. The system of claim 2 wherein the conventional ventilation pattern includes one or more of the following techniques:
controlled mechanical ventilation; assisted mechanical ventilation; assisted mechanical ventilation with trigger systems for detecting the patient spontaneous activity and synchronizing the delivery of inspiratory or expiratory pressure to the patient's spontaneous breaths.

7. The system of claim 1, wherein the breathing circuit includes an aninspiratory line and a separated exhalation line.

8. The system of claim 1, wherein the breathing circuit includes a singletube and an intentional leak allowing gas purging at the patient's airway opening during the expiratory phase.

9. A method for operating the system of claim 1, comprising steps of:
detecting by means of at least one sensor the alternation of the inspiration and the expiration phases in a patient; and
controlling the pressure generator means to provide the high frequency ventilation to the patient only during the expiration phase.

10. The method of claim 9 wherein high frequency percussive ventilation is delivered to the patient over a full expiratory phase, i.e. during an initial drop in lung volume until a predetermined Positive End Expiratory Pressure is reached, and kept until a next inspiratory cycle is initiated.

11. A computer program for causing the system of claim 1 to perform a method when the computer program is executed on a computer, the method comprising steps of:
detecting by means of at least one sensor the alternation of the inspiration and the expiration phases in a patient:
controlling the pressure generator means to provide the high frequency ventilation to the patient only during the expiration phase.

12. A system according to claim 1 for use in a method to treat a patient suffering respiratory failures.
